(19) United States
(12) Patent Application Publication
(10) Pub. No.: US 2007/0166414 A1
(43) Pub. Date: Jul. 19, 2007

(54) METHOD FOR ENHANCING TAMOXIFEN EFFICACY AS A CANCER THERAPEUTIC

(76) Inventor: Andreas Constantinou, Nicosia (CY)

Correspondence Address: DONNA J. RUSSELL, 1492 ANTHONY WAY, MT. JULIET, TN, 37122, US

(21) Appl. No.: 11/623,089

(22) Filed: Jan. 14, 2007

Related U.S. Application Data
(60) Provisional application No. 60/758,653, filed on Jan. 13, 2006.

(51) Int. Cl. A61K 36/48; A61K 31/7072; A61K 31/7048; A61K 31/704; A61K 31/4745; A61K 31/404; A61K 31/337

(52) U.S. Cl. 424/757; 514/27; 514/34; 514/283; 514/109; 514/449; 514/49; 514/251; 514/414; 514/456

(57) ABSTRACT

Disclosed is a method for enhancing the effectiveness of tamoxifen in the prevention and treatment of hormone-associated cancer such as breast cancer. Also disclosed is a method for decreasing the risk of endometrial hyperplasia and endometrial cancer associated with tamoxifen therapy. In the method, tamoxifen therapy is combined with administration of daidzein or its equol metabolite.

### CROSS-REFERENCE TO RELATED APPLICATIONS

This application claims the benefit of priority of earlier-filed U.S. Provisional Patent Application No. 60/758,653.

### FIELD OF THE INVENTION

The invention relates to compositions and methods for treating cancer. More specifically, the invention relates to compositions and methods for increasing the efficacy of the anti-cancer agent tamoxifen.

### BACKGROUND OF THE INVENTION

In 1998, the National Surgical Adjuvant Breast and Bowel Project (NSABP) demonstrated that tamoxifen treatment reduced the incidence of both invasive and non-invasive breast cancer in women at high risk for the disease. Following the results of this study, women at high risk for developing breast cancer have been prescribed tamoxifen to prevent primary breast cancer. Tamoxifen is also prescribed to those women with estrogen receptor (ER) positive breast cancer to prevent secondary tumors. Tamoxifen works by blocking estrogen receptors and preventing estrogen from attaching to them. It is generally used after surgery or radiation therapy in women who are past menopause because it reduces the risk that the cancer will return. Tamoxifen may also used by some women without breast cancer but who are at high risk of developing it, because it helps lower the risk.

Soy products, containing phytoestrogens (including primarily the isoflavones genistein and daidzein), have become popular dietary supplements among women in Western societies due to their proposed inhibition of breast cancer, cardiovascular disease, and postmenopausal symptoms. At relatively high concentrations and in vito, genistein has been shown to inhibit enzymatic activities crucial for tumor cell proliferation, such as the protein tyrosine kinases and topoisomerase II. Genistein has also been found to be effective in preventing DMBA-induced mammary tumorigenesis in female rats, but only when administered to neonatal or prepubescent rats younger than 35 days old.

Studies have indicated, however, that genistein may compete with tamoxifen for the estrogen receptors and, consequently, the consumption of soy products can reduce the efficacy of tamoxifen. In one study conducted in athymic mice, genistein negated the inhibitory effect of tamoxifen on the growth of implanted oestrogen-dependent MCF-7 cells and researchers discovered that when taken as part of a daily diet, genistein can inhibit the ability of tamoxifen to halt breast cancer growth (Cancer Research 62: 2474-2477). Based on these results, medical practitioners in the United States generally suggest that women for whom tamoxifen has been prescribed avoid consuming soy products or taking soy isoflavone supplements.

Reproductive cancer is the third most common type of cancer in women, with only lung and breast cancer occurring at higher numbers. Endometrial hyperplasia is believed to represent the precursor lesion for type I endometrial carcinoma. Continued estrogenic stimulation unopposed by progesterone often leads to endometrial hyperplasia, which is characterized by an overgrowth of glandular component in favour of the supportive stroma. Estrogen exposure is associated with an increase in the incidence of breast cancer and various uterine lesions including tumors. It has been reported that in up to 83% of endometrial cancers there is a mutational inactivation of the tumor suppressor gene PTEN. For this reason PTEN is characterized as a “gatekeeper” for endometrial cancer. Although tamoxifen is among the least toxic anticancer agents, its long term administration may increase the risk of endometrial cancer and benign endometrial pathologies. Tamoxifen, acting as an estrogen agonist in the uterus, promotes cell proliferation, increases PCNA expression and induces DNA damage in the tissue.

What are needed are therapeutic agents that may be used in conjunction with tamoxifen to increase tamoxifen's efficacy in treating breast cancer without increasing the risk of endometrial cancer associated with tamoxifen therapy.

### SUMMARY OF THE INVENTION

The present invention relates to a method for treating estrogen-associated (hormone-associated) cancer, the method comprising administering a therapeutic dosage of a composition chosen from the group consisting of soy germ, soy germ extract, isolated or synthetic daidzein, natural or synthetic equol, functionally equivalent derivatives or analogs of daidzein or equol, or combinations thereof to a patient undergoing a tamoxifen treatment regimen. In one embodiment, the hormone-associated cancer is breast cancer.

In one aspect, the method also comprises administering an additional chemotherapeutic agent such as a composition chosen from the group consisting of paclitaxel, adriamycin, cyclophosphamide, capecitabine, vinorelbine, carboplatin, epirubicin, docetaxel, methotrexate, 5-fluorouracil, anastrozole, letrozole, exemestane, or combinations thereof.

The invention also provides a method for increasing the efficacy of tamoxifen therapy in the prevention of hormone-associated cancer in a human patient, the method comprising administering a therapeutically effective amount of a composition chosen from the group consisting of soy germ, soy germ extract, isolated or synthetic daidzein, natural or synthetic equol, functionally equivalent derivatives or analogs of daidzein or equol, or combinations thereof to a patient undergoing a tamoxifen treatment regimen.

In another aspect, the invention provides a method for decreasing the risk of endometrial hyperplasia or endometrial cancer associated with tamoxifen therapy, the method comprising administering a therapeutically effective amount of a composition chosen from the group consisting of soy germ, soy germ extract, isolated or synthetic daidzein, natural or synthetic equol, functionally equivalent derivatives or analogs of daidzein or equol, or combinations thereof to a patient undergoing a tamoxifen treatment regimen.

### BRIEF DESCRIPTION OF THE DRAWINGS

FIG. 1 is a graph illustrating the effect of experimental diets, on tumor incidence (A) and mean number of tumors (B) in female Sprague-Dawley rats exposed to dimethylbenzanthracene (DMBA). Rats (n=20) were started on basal AIN-76A diet (black) or diets containing tamoxifen (TAM) (yellow), genistein (GEN) (blue), TAM/GEN (green), daidzein (DAI) (red), or TAM/DAI (orange) one week prior to administration of DMBA. Rats were maintained on the diet throughout the experiment. Tumors were detected by palpation during the study. *, P<0.05 vs. +control. **, P<0.01 vs. +control. ***, P=0.001

FIG. 2 is a graph illustrating the effect of experimental diets on mean latency (A) and tumor burden (B) of mammary tumors in female Sprague-Dawley rats exposed to dimethylbenzanthracene. Latency was determined by the first appearance of palpable tumors in the rats. Data are means with SD shown as T-bars (n=20). Statistical comparison of tumor latency between groups was by unpaired t-test. Abbreviations: TAM, tamoxifen; GEN, genistein; DAI, daidzein. Statistical significance is as follows: *, P<0.05 vs. +control group. **, P<0.01 vs. +control group. ***, P<0.001 vs. +control group. ###, P<0.001 TAM/DAI group vs. TAM group.

FIG. 3 is a graph illustrating results of LC-MS-MS analysis of 8-oxo-deoxyguanosine (8-oxo-dG) from the normal mammary glands of female Sprague-Dawley rats fed control or experimental diets. All groups were exposed to dimethylbenzanthracene except−control. Data representing the ratio of 8-oxo-dG to deoxyguanosine (dG) are means of triplicate samples with SD shown as T-bars (n=6). Statistical significance is as follows: *, P<0.05 vs. +control group. **, P<0.01 vs. +control group; #, P<0.05 TAMIDAI group vs. −control. Abbreviations: TAM, tamoxifen; GEN, genistein; DAI, daidzein.

FIG. 4 is a series of chemical structures provided as examples of equol derivatives and analogs.

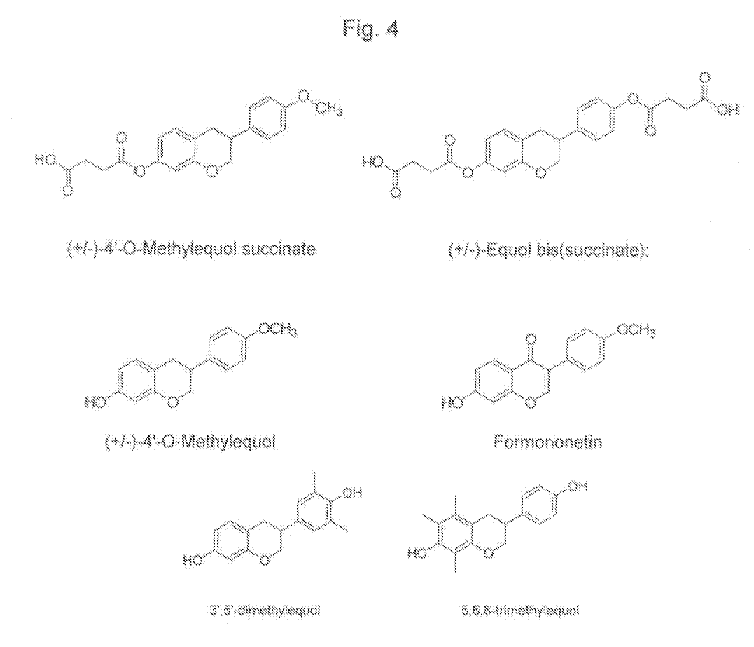

FIG. 5 is a bar graph illustrating DNA damage as determined by comet assay. Bars represent the average of 18 measurements from each group. Tamoxifen provided in the diet of rats in the study significantly increased DNA damage as compared to that caused by the basal diet. Daidzein had no damaging effect. Daidzein provided in combination with tamoxifen decreased the level of DNA damage associated with tamoxifen alone.

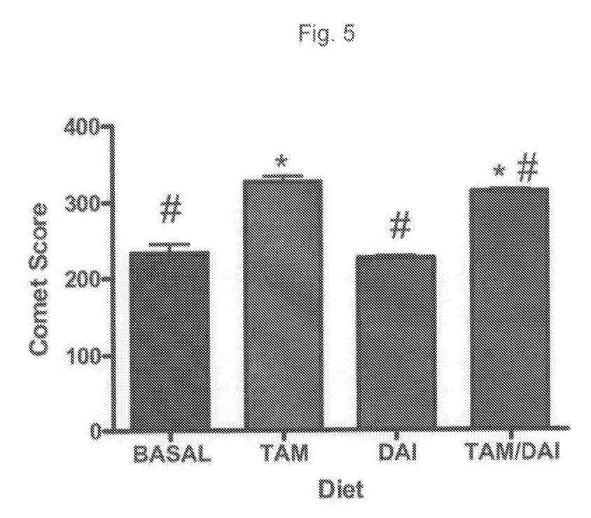

FIG. 6a provides photographs of PTEN staining of normal endometrial tissue (negative control) and mammary tumor tissue (positive control). FIG. 6b is a series of photographs of PTEN staining of tissues taken from rats fed the indicated diets for 28 days. FIG. 6c is a series of photographs of PTEN staining of tissues taken from rats fed the indicated diets for 185 days. In both the shorter-term and longer-term studies, tamoxifen alone or daidzein alone produced a reduction in PTEN expression. The combination of daidzein and tamoxifen, however, had no effect on PTEN expression.

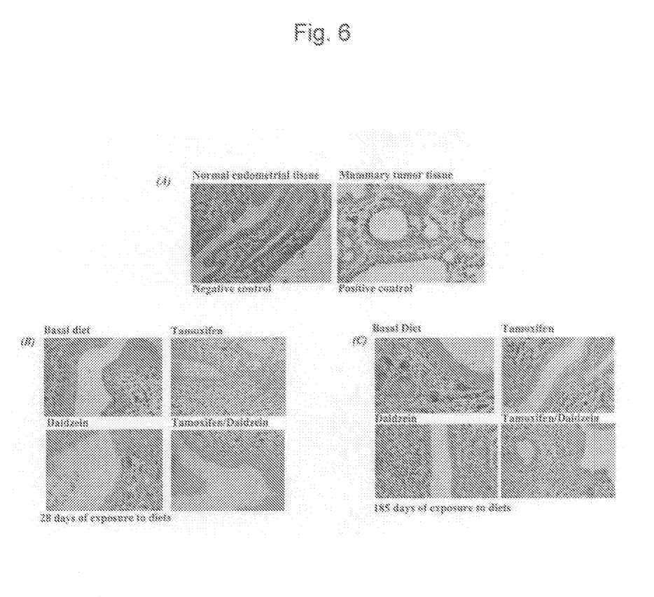

FIG. 7 provides photographs of PCNA staining of endometrial tissue negative control and positive control.

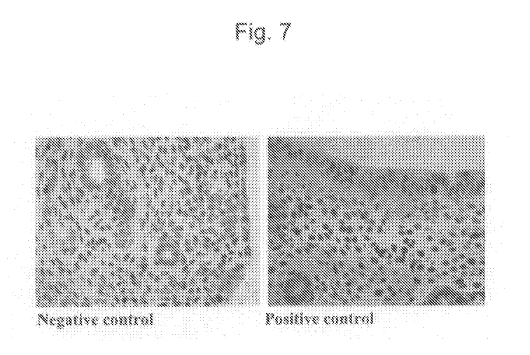

FIG. 8 is a series of photographs of PCNA staining of tissues taken from rats fed the indicated diets for 28 days.

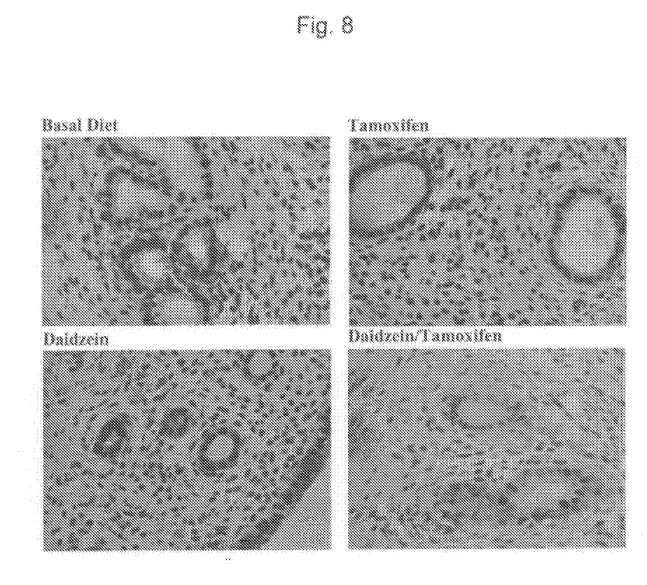

FIG. 9 is a bar graph illustrating percentage expression of PCNA in tissues from rats fed for 28 days the diets indicated on the x axis.

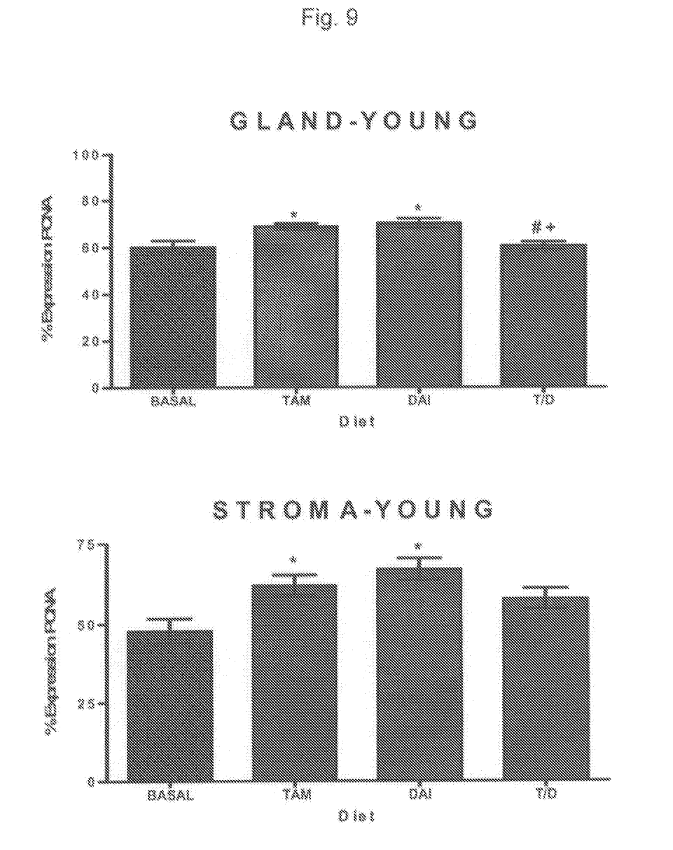

FIG. 10 is a series of photographs of PCNA staining of tissues taken from rats fed the indicated diets for 185 days.

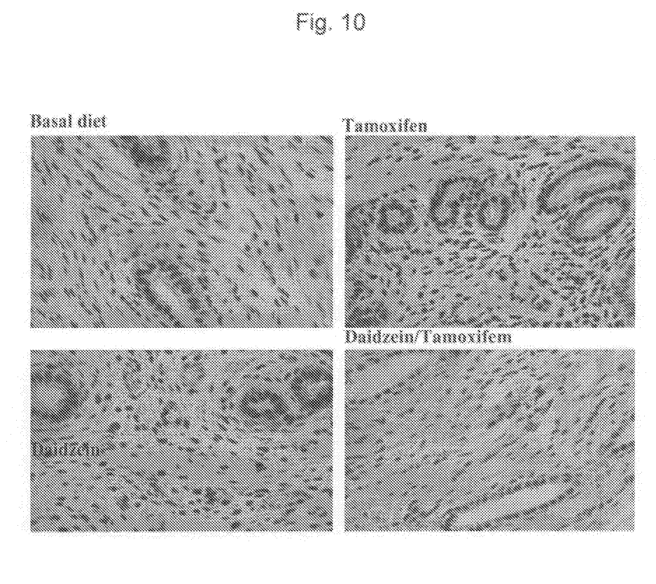

FIG. 11 is a bar graph illustrating percentage expression of PCNA in tissues from rats fed for 185 days the diets indicated on the x axis. As illustrated in FIGS. 7-11, in gland and stroma tissue of rats fed tamoxifen alone or daidzein alone there is increased expression of PCNA compared to the corresponding tissues of rats fed the basal diet. Interestingly, the daidzein/tamoxifen combination diet reduces the expression of PCNA in both tissues to the levels of the basal diet.

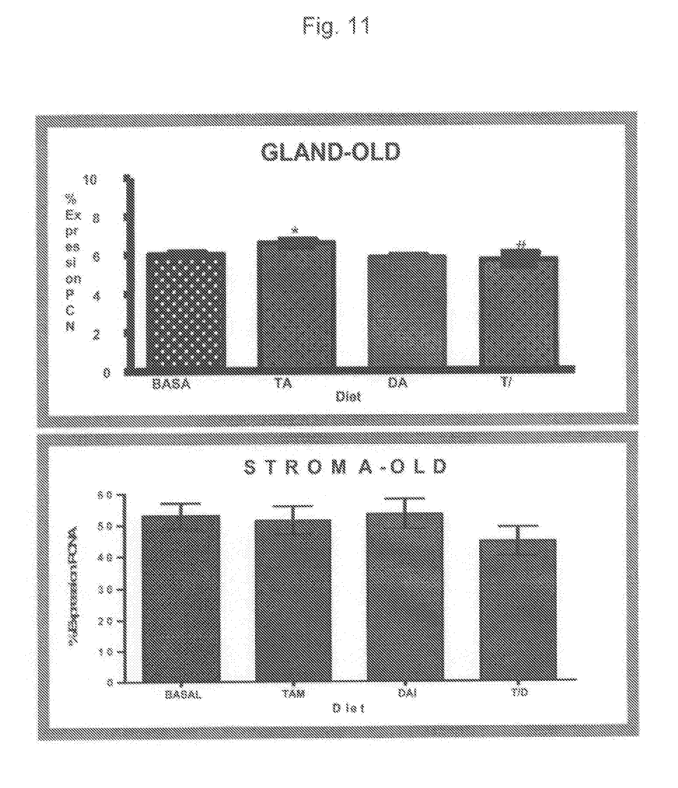

### DETAILED DESCRIPTION

The inventors have discovered that isolated daidzein (7-hydroxy-3-(4-hydroxyphenyl)-4H-1-benzopyran-4-one; 4′, 7-dihydroxyisoflavone), its major metabolite equol (7-hydroxy-3-(4′-hydroxyphenyl)-chroman), and analogs of equol can be provided in combination with tamoxifen to increase the effectiveness of tamoxifen and to prevent certain unwanted side-effects of tamoxifen therapy. Provided as part of the diet, a tamoxifenldaidzein combination has been shown to reduce mammary tumor multiplicity by 76%, tumor incidence by 35%, tumor burden by over 95%, and to increase tumor latency by 62%, as compared to a control diet. The diet significantly decreased 8-oxo-deoxyguanosine levels (an indicator of oxidative DNA damage) in the mammary glands. The inventors have also demonstrated that the equol metabolite of daidzein is the effective agent, and therefore isolated equol, an equol analog, or a synthetic form of equol in combination with tamoxifen may also provide the desired increase in tamoxifen benefit.

Tamoxifen is an effective chemopreventive agent against estrogen receptor positive breast cancer. Although the long term administration of tamoxifen substantially reduces the risk of breast cancer, it also significantly increases the risk of endometrial cancer, as well as the incidence of benign endometrial pathologies. The inventors found that the combination of tamoxifen with daidzein prevented 7, 12, dimethylbenz[a]-anthracene (DMBA)-induced rat mammary carcinogenesis in female Sprague-Dawley rats more effectively than tamoxifen alone, Since daidzein may be converted to equol in the rats gut and is found in the blood in this form they attributed the effect of daidzein to the equol metabolite. They also discovered that equol diminishes the risk for endometrial cancer induced by tamoxifen. Their data demonstrates for the first time that the soy isoflavone daidzein reduces the risk of endometrial cancer in female rats.

In one of the inventors'studies, female Sprague-Dawley rats were placed on diets supplemented with tamoxifen, genistein, daidzein, or a combination of each isoflavone with tamoxifen. A week later mammary tumors were induced by 7, 12-dimethylbenzanthracene (DMBA). The tamoxifen/daidzein combination reduced tumor multiplicity by 76%, tumor incidence by 35%, tumor burden by over 95%, and increased tumor latency by 62% compared to the positive control. The tamoxifendaidzein combination diet was in all measured aspects more effective while the tamoxifen/genistein combination was less effective than the tamoxifen diet. The tamoxifen/daidzein diet significantly decreased 8-oxo-deoxyguanosine levels in the mammary glands.

Miso (containing soy isoflavones) in combination with tamoxifen has been shown to be more effective than tamoxifen alone in preventing N-nitroso-N-methylurea-induced rat mammary cancers. The inventors have also found that tamoxifen combined with soy protein isolate provides more protection against DMBA-induced mammary carcinogenesis in rats than it does by itself. The inventors'goal was therefore to identify the component(s) of soybeans that work together with tamoxifen to prevent mammary tumors in female Sprague-Dawley rats and to investigate the potential mechanisms of action. What they have discovered is that the soy isoflavone that promotes the beneficial effects of tamoxifen while limiting its unwanted side-effects is daidzein and its metabolite equol.

Soy germ and soy germ extract contain higher amounts of daidzein and little genistein. The invention therefore comprises a method of using compositions comprising soy germ, soy germ extract, isolated or synthetic daidzein, natural or synthetic equol, functionally equivalent derivatives or analogs of daidzein or equol, or combinations thereof in combination with tamoxifen for the treatment of cancer. Compositions comprising soy germ, soy germ extract, or a combination thereof may be provided as dietary supplements, for example, to be consumed by a patient either concurrently or sequentially with administration of tamoxifen. For sequential administration, a composition comprising soy germ, soy germ extract, or a combination thereof may be consumed either prior to or following tamoxifen administration, providing a therapeutically effective level of daidzein, and its equol metabolite, to increase the effectiveness of the tamoxifen administered. Administration, as used herein, may be administration of the appropriate agent by a heathcare practitioner to a patient or may be self-administration by the patient.

Compositions comprising isolated or synthetic daidzein, isolated or synthetic equol, functionally equivalent derivatives or analogs of daidzein or equol, or combinations thereof may be provided as either pharmaceutical agents in combination with tamoxifen or as nutritional supplements for consumption by a patient during the course of tamoxifen administration. A treatment regimen of the invention may also include consumption of soy germ, soy germ extract, isolated or synthetic daidzein, isolated or synthetic equol, functionally equivalent derivatives or analogs of daidzein or equol, or combinations thereof prior to and/or following the completion of a tamoxifen treatment regimen. Approximately 30% of humans metabolize daidzein to equol. Therefore, compositions comprising equol alone, or in combination with daidzein, may be particularly effective for use in the method of the invention. Method for synthesizing equol have been described previously (e.g., J. Nat. Products (1995) 58(12): 1901-1905; U.S. patent application Ser. No. 11/566,569, Hyatt). “Natural” equol may be obtained, for example, from the biosynthesis of equol from daidzein in a bacterial system.

Compounds used in the method of the invention can be formulated as pharmaceutical compositions comprising daidzein, equol, or a combination thereof, optionally additionally comprising tamoxifen to provide either a daidzein and/or equol composition that may be taken or administered in a separate dosage form from the tamoxifen or a daidzein and/or equol composition comprising a dosage form with tamoxifen, and administered to a patient in a variety of dosage forms adapted to the chosen route of administration (e.g., orally or parenterally, by intravenous, intramuscular, topical or subcutaneous routes). They may be systemically administered orally, for example, in combination with a pharmaceutically acceptable vehicle such as an inert diluent or an edible carrier. They may be enclosed in hard or soft shell gelatin capsules, may be compressed into tablets, or may be incorporated directly with the food of a patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound.

Tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; lubricants such as magnesium stearate; and one or more sweetening agents such as sucrose, fructose, lactose or aspartame. A flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor.

Compositions comprising isolated or synthetic daidzein, isolated or synthetic equol, functionally equivalent derivatives or analogs of daidzein or equol, or combinations thereof may also be incorporated into sustained-release preparations and devices.

One or more of the compositions may also be administered intravenously, subcutaneously, or intraperitoneally by infusion or injection. Solutions of the active compound or its salts can be prepared in water, optionally mixed with a nontoxic surfactant. Dispersions can also be prepared in glycerol or liquid polyethylene glycols, for example, and mixtures thereof and in oils. Under ordinary conditions of storage and use, these preparations may contain a preservative to prevent the growth of microorganisms.

Pharmaceutical dosage forms suitable for injection or infusion can include sterile aqueous solutions or dispersions or sterile powders comprising the active ingredient which are adapted for the preparation of sterile injectable or infusible solutions or dispersions, optionally encapsulated in liposomes. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants.

The method of the invention may also comprise administering other chemotherapeutic drugs or hormonal therapies including, but not limited to, paclitaxel, adriamycin, cyclophosphamide, capecitabine, vinorelbine, carboplatin, epirubicin, docetaxel, methotrexate, 5-fluorouracil, anastrozole, letrozole, and exemestane.

The method described by the invention increases the effectiveness of tamoxifen while decreasing its side-effects. It therefore provides treatment options that include decreasing tamoxifen dosage because of the increased effectiveness of a combination of at least one daidzein and/or equol composition and tamoxifen as well as increasing tamoxifen dosage, if needed, because the combination of at least one daidzein and/or equol composition and tamoxifen decreases the risks associated with tamoxifen therapy.

The invention may be further described by means of the following non-limiting examples.

### EXAMPLES

Materials and Methods

Chemicals and Tissue Culture. All chemicals, unless specified otherwise, were purchased from Sigma Chemical Co. (St. Louis, Miss. USA). Genistein and daidzein were purchased from Indofine Chemical Co. (Somerville, N.J. USA). All tissue culture components were obtained from Invitrogen Corp. (Carlsbad, Calif. USA) unless otherwise stated.

Chemoprevention Study. The diets were formulated by Harlan-Teklad (Madison, Wis. USA) to contain approximately the same amounts of protein, fat, and carbohydrates. The basal diet AIN-76A is free of soy products. The diets were also adjusted to contain similar amounts of methionine, cystine, and choline because these nutrients have been reported to affect mammary carcinogenesis. Daidzein and genistein were added in the basal diets as necessary (Table 1), at 140 and 105 mg/Kg of diet respectively. The selected isoflavone concentrations were based on the amounts present in the well-tolerated 16% (w/w) S PI diet (13). The tam oxifen dose of 0.125 mg/Kg diet was selected based on a previous study (24).

Virgin female Sprague-Dawley rats at 35 days of age were randomized by weight into 5 groups (20 rats per group) and fed basal AIN-76A diet. At 43 days of age, all but the control groups received basal diet supplemented with the appropriate test agents. After an additional week the animals received a single dose (12.5 mg) of DMBA intragastrically in sesame oil.

During the experimental period, the animals were weighed weekly. Palpation of mammary tumors began four weeks after animals received DMBA and continued until termination of the study. The date of appearance and location of every palpable tumor were recorded. Rats were observed daily to assess their general health. Rats were sacrificed by CO₂ asphyxiation. Mammary tumors were coded by location, removed from the rat, and weighed. Sections of normal mammary tissue were also taken and either flash frozen in liquid nitrogen or formalin-fixed for paraffin embedding. The experiment and protocol was approved and performed in compliance with relevant laws and institutional guidelines according to the Animal Care Committee (01-142) and Animal Welfare Assurance (A3460.01) at the University of Illinois at Chicago.

DNA Hydrolysis. DNA was extracted from normal mammary tissue from six different rats from each group and dissolved in water at ˜2 mg/mL. Aliquots (50-μL) containing ˜100 μg of DNA were mixed with 50 μL of buffer (50 mM ammonium acetate, 0.2 mM ZnCl₂, pH 5.3), 10 μL of nuclease P1 (0.4 unit/μL), and 8 μL of alkaline phosphatase (1 unit/μL in 10 mM Tris pH 7.4) and incubated at 37° C. for 30 minutes. For DNA oxidation detection, a 60-μL aliquot of the DNA digest and 10 μL of internal standard mixture (containing 3.0 ppb [¹³C₁₃ ¹⁵N₅]-8-oxo-dG and 1.5 ppb [¹³C₁₀, ¹⁵N₅]-8-oxo-dA) were combined in 30,000 molecular weight cut-off ultrafiltration centrifuge tube (Amicon; Bedford, Mass. USA). The solution was centrifuged for 15 minutes at 12,000 rpm and 4° C. just before LC-UV-MS-MS analysis.

LC-UV-MS-MS Quantification of Oxidized Nucleosides. The HPLC system consisted of a ThermoFinnigan (San Jose, Calif. USA) Surveyor MS pump coupled with an autosampler, a PDA detector and a YMC (Wilmington, N.C. USA) ODS-AQ C₁₈ column (2.0×250 mm, 5 μm), and guard column (4.0×20 mm). Absorbance detection at 240-290 nm was used for on-line HPLC quantification of dG and dC in the DNA hydrolysate, and a standard curve was prepared using standard solutions of dG and dC in the initial mobile phase. The flow rate was 200 μL/min, and the injection volume was 20 μL. For DNA oxidation, the solvent system consisted of water/methanol 90:10 (v/v) at the start point and a linear gradient from 10 to 25% (v/v) methanol over 0-25 min. Then methanol increased to 90% (v/v) in 30 sec. After that the mobile phase was held at 90% (v/v) methanol for 7.5 min.

Selected reaction monitoring (SRM) was carried out using a TSQ Quantum triple quadruple mass spectrometer equipped with negative ion (for DNA oxidation) electrospray ionisation. Nitrogen was used as the sheath gas and auxiliary gas at 25 and 10 arbitrary units, respectively. The spray voltage was 30 eV. Collision-induced dissociation (CID) was carried out using argon at 1.0-1.5 mTorr with the collision energy of 10-20 V. The span time per ion channel was 0.5 seconds during SRM. For 8-oxo-deoxyguanosine (8-oxo-dG), m/z 282>192 was selected as SRM (m/z 297>204 for the isotopically labelled internal standard). For 5-MedC, m/z 242>126 was selected as SRM (m/z 245>129 for the isotopically labelled internal standard).

LC-MS analysis of isoflavone levels. Serum isoflavone levels of animals on the diets for four weeks without DMBA treatment were analysed. Blood was collected by orbital bleeding. Serum (400 μL) from each sample was transferred into a 2-mL Eppendorf tube. To each tube, 100 μL of 1 M (pH 5.0) buffer was added followed by vortex mixing, then 40 μL sulphatase (50 mg/mL, Sigma # S-3009) enzyme solution was added. After shaking, the mixture was incubated overnight in a 37° C. water bath (>17 h). The enzymatic hydrolysis was stopped by adding 1 mL ice-cold ethanol/acetonitrile (1:1; v/v), mixed and centrifuged at 4° C. for 15 minutes (10,000 rpm). The supernatant was removed and evaporated to dryness under vacuum. The resulting residue was reconstituted with 150 μL internal standard solution, and three aliquots (15 μL each) were analysed using LC-MS (HP 1100 LC-MS D) over an Xterra RP-18 column (2.1×100 mm, 3.5 μm). The mobile phase was 0.1% (v/v) formic acid followed by acetonitrile (containing 0.1% (v/v) formic acid). Elution was at 200 μL/min. The MSD was operated in negative electrospray ionisation mode with selected ion monitoring of m/z 253 for daidzein and internal standard (chrysin), m/z 269 for genistein, and m/z 241 for equol. Three to four samples from each group were analysed and resulting values are mean± SD.

Statistical analysis. A significant inhibition of tumor induction as achieved by administration of an inhibitor was defined as a statistically significant decrease (at 5% level) in tumor incidence, multiplicity, or latency period. Statistical comparison of tumor latency between groups was by unpaired t-test. The statistical significance of differences between mean tumor multiplicities was assessed using analysis of variance (ANOVA), and the Armitage'stest for trends in proportions (25). Tumor incidence rates were generated by the life table method, and compared by (one tailed) log-rank analysis (25).

Results

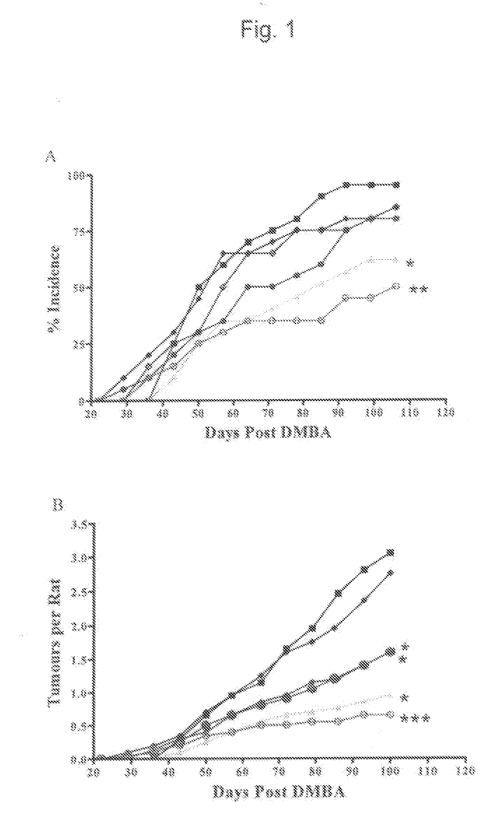

Effects of diets on tumor incidence, multiplicity, latency and burden. The effects of the experimental diets on tumor incidence and multiplicity over the length of the study (114 days) are shown in FIG. 1. A decrease in tumor incidence rate was evident in all groups fed the experimental diets in comparison to the group fed the basal AIN76A diet and received DMBA (+control). The effect of genistein or daidzein containing diets on the tumor incidence rate was statistically non-significant (P=0.408, P=0.366, respectively, FIG. 1A and Table 1). Tamoxifen produced a significant reduction (P=0.015) and the tamoxifen/daidzein combination diet was most effective at reducing incidence rate (P=0.005). Daidzein improved the effect of tamoxifen with respect to tumor incidence whereas genistein weakened it, and the tamoxifen/genistein combination failed to produce a significant reduction in tumor incidence rate in comparison to the control (P=0.823). All diets containing tamoxifen produced a small effect on body weight, but it remained within 94% of the control value (Table 1).

Tumor multiplicity was significantly reduced in groups fed tamoxifen (P=0.012), daidzein (P 0.049), tamoxifen/daidzein (P=0.001) and tamoxifen/genistein (P =0.029). Thus, daidzein improved and genistein reduced the effect of tamoxifen with respect of tumor multiplicity (FIG. 1b and Table 1).

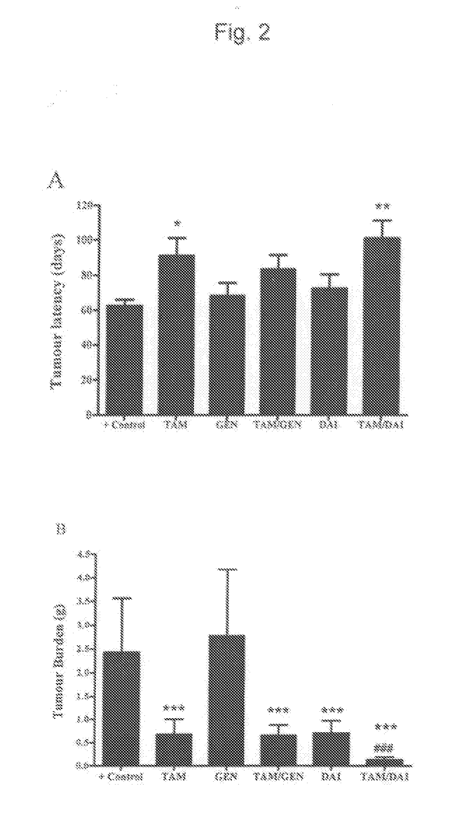

Tumor latency increased in all experimental groups in comparison to the control group (FIG. 2a and Table 1). However, the increase was statistically significant only in the tamoxifen (P=0.013), and tamoxifen/daidzein (P=0.002) groups (Table 1). The increase over the control value in the tamoxifen group was 46% compared to 62% in the tamoxifen/daidzein group and 18% in the ta moxifen/genistein group. Therefore, daidzein improved the effect of tamoxifen on tumor latency and genistein curtailed it.

A similar effect was evident in tumor burden (FIG. 2b), defined as the average tumor weight per group. The mean tumor weight in the control group was 2.5 g but ranged greatly from 0.01 g to 14 g. Daidzein reduced tumor burden as effectively as tamoxifen while genistein proved ineffective. The tamoxifen/daidzein and tamoxifen/genistein combinations also reduced tumor burden significantly in comparison to the control value. The reduction in tumor burden by the tamoxifen/daidzein group was statistically significant even in comparison to tamoxifen (P<0.001). Therefore, the combination of genistein with tamoxifen neither improved nor attenuated the effectiveness of tamoxifen on average tumor burden, whereas a substantial improvement was evident in the group fed the tamoxifenldaidzein combination diet.

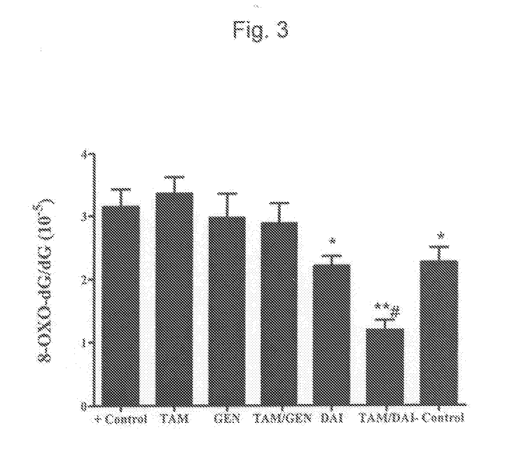

Effect of diets on oxidative DNA damage and serum isoflavone levels. LC-UV-MS-MS was used to determine oxidative DNA damage in the mammary glands, a method known to be highly selective and sensitive. This is the most suitable method for detecting the oxidation product 8-oxo-deoxyguanosine (8-oxo-dG), considered to be one of the most critical lesions leading to carcinogenesis. The effect of the experimental diets on the ratio of 8-oxo-dG/dG was compared to that of the control diets, and the results are shown in FIG. 3. As expected from an earlier report (26) the 8-oxo-dG levels in the DMBA group (+control) were higher than the group not exposed to DMBA (−control). Tamoxifen, genistein, and tamoxifen/genistein diets were ineffective in suppressing these DMBA-induced DNA alterations. However, daidzein significantly (P<0.05) suppressed the levels of 8-oxo-dG equal to those of the group never exposed to DMBA (−control). The tamoxifen/daidzein combination markedly lowered the damage with respect to the +control (P<0.001). More importantly, the tamoxifen/daidzein diet lowered oxidative DNA damage to the levels below the−control group (P<0.05). This finding suggests the tamoxifen/daidzein combination not only blocks DMBA initiated oxidative DNA damage but it also reduces endogenous oxidative DNA damage. Thus, both diets containing daidzein are effective against oxidative DNA damage, as determined by measuring the 8-oxo-dG levels.

The genistein, daidzein, and equol isoflavone levels were measured in the serum of 3-4 animals from groups on the same diets for four weeks and were never exposed to DMBA. As expected, the control (basal diet) and tamoxifen groups had very low levels of genistein and daidzein. The genistein and tamoxifen/genistein groups showed very low levels of daidzein, the daidzein and tamoxifen/daidzein groups had very low levels of genistein. Equol, the end product of the metabolic pathway of daidzein (19), was also measured in the serum of the rats not exposed to DMBA. The equol levels in the control, tamoxifen, genistein, and tamoxifen/genistein groups were not detectable, although these groups contained small quantities of genistein and daidzein (Table 2). Equol was present at very high concentrations in the daidzein and tamoxifen/daidzein groups, although daidzein itself was present at much lower concentrations (Table 1), suggesting the vast majority of daidzein had been metabolised to equol.

Four groups of female rats were fed the basal AIN-76A diet supplemented with: 1) vehicle (control), (2) tamoxifen (0.125 mg/Kg), (3) daidzein (140 mg/Kg), (4) tamoxifen (0.125 mg/Kg) plus daidzein (140 mg/Kg) combination. Rats were sacrificed at either 28 days (short term exposure) or 185 days (long term exposure) after diet initiation, and the endometrium was removed and processed for immunohistochemical and biochemical evaluations. The inventors determined that the most significant differences were within the glandular cells of the endometrium. In these cells, both short-term and long-term exposure to the tamoxifen diet produced increased DNA damage, increased proliferating cell nuclear antigen (PCNA) expression and reduced PTEN expression. The combined tamoxifen/daidzein diet reversed these effects to a large degree. The diadzein alone did not affect the level of DNA damage when compared to the control diet whereas it increased PCNA and reduced PTEN expression.

TABLE 1
Composition of diets and their effects on final tumor incidence, incidence rate, multiplicity,
survival, and body weight.

| | Added diet | Final tumor | Incidence | Multiplicity | Latency | Survival | Mean body |
|---|---|---|---|---|---|---|---|
| Group | component | incidence¹ | rate | (P-value)³ | (P- | (%) | weight |
| +Control | none | 95 | — | — | — | 90 | 100.0 |
| (DMBA) | | | | | | | |
| TAM | 0.125 | 73* | 0.015 | 0.012 | 0.013 | 90 | 94.4 |
| GEN | 140.0 | 85 | 0.408 | 0.596 | 0.326 | 95 | 100.7 |
| DAI | 105.0 | 85 | 0.366 | 0.049 | 0.322 | 95 | 99.2 |
| TAM/GEN | 0.125/140.0 | 79 | 0.823 | 0.029 | 0.280 | 89 | 96.3 |
| TAM/DAI | 0.125/105.0 | 60** | 0.005 | 0.001 | 0.002 | 100 | 94.4 |

Abbreviations:
DMBA, dimethylbenzanthracene;
TAM, tamoxifen;
GEN, genistein;
DAI, daidzein
All Statistical comparisons were made for experimental groups (n = 20) versus control (DMBA) group.
¹Comparisons were made by Fisher's exact test. Statistical significance is as follows: *P < 0.05; **P < 0.01.
²Comparisons of data shown in FIG. 1A with log-rank analysis (26)
³Comparison of data shown in FIG. 1B with one-tailed test for trends in proportions.
⁴Comparisons made by unpaired t-test

TABLE 2
Mean serum levels (ng/ml) ± SEM¹ of soy isoflavones in
female rats fed the control and experimental diets for four weeks.

| Diet | GEN | DAI | Equol |
|---|---|---|---|
| Basal (control) | 1.2 ± 0.1 | 0.2 ± 0.1 | 0.0 |
| TAM | 0.8 ± 0.1 | 0.2 ± 0.0 | 0.0 |
| GEN | 116.3 ± 11.3 | 0.8 ± 0.2 | 0.0 |
| TAM/GEN | 119.7 ± 2.6 | 1.0 ± 0.1 | 0.0 |
| DAI | 1.2 ± 4.7 | 3.6 ± 0.9 | 115.0 ± 20.6 |
| TAM/DAI | 3.9 ± 1.0 | 3.6 ± 0.4 | 148.0 ± 16.5 |

Abbreviations:
TAM, tamoxifen;
GEN, genistein;
DAI, daidzein
¹Serum was obtained from 3-4 rats from each group and LC-MS analysis was performed in triplicate for each serum sample. The MSD was operated in negative electrospray ionization mode with selected ion monitoring of m/z 253 for DAI and m/z 241 for equol as described in the Materials and Methods section.

What is claimed is:

1. A method for treating hormone-associated cancer, the method comprising administering a therapeutic dosage of a composition chosen from the group consisting of soy germ, soy germ extract, isolated or synthetic daidzein, natural or synthetic equol, functionally equivalent derivatives or analogs of daidzein or equol, or combinations thereof to a patient undergoing a tamoxifen treatment regimen.

2. The method of claim 1 wherein the composition is chosen from the group consisting of isolated or synthetic daidzein, natural or synthetic equol, functionally equivalent derivatives or analogs of daidzein or equol, or combinations thereof.

3. The method of claim 1 wherein the composition comprises natural or synthetic equol.

4. The method of claim 1 further comprising administering a non-tamoxifen chemotherapeutic agent.

5. The method of claim 4 wherein the chemotherapeutic agent is chosen from the group consisting of paclitaxel, adriamycin, cyclophosphamide, capecitabine, vinorelbine, carboplatin, epirubicin, docetaxel, methotrexate, 5-fluorouracil, anastrozole, letrozole, exemestane, or combinations thereof.

6. A method for increasing the efficacy of tamoxifen therapy in the prevention of hormone-associated cancer in a human patient, the method comprising administering a therapeutically effective amount of a composition chosen from the group consisting of soy germ, soy germ extract, isolated or synthetic daidzein, natural or synthetic equol, functionally equivalent derivatives or analogs of daidzein or equol, or combinations thereof to a patient undergoing a tamoxifen treatment regimen.

7. The method of claim 6 wherein the composition is chosen from the group consisting of isolated or synthetic daidzein, natural or synthetic equol, functionally equivalent derivatives or analogs of daidzein or equol, or combinations thereof.

8. The method of claim 6 wherein the composition comprises natural or synthetic equol.

9. The method of claim 6 further comprising administering a non-tamoxifen chemotherapeutic agent.

10. The method of claim 9 wherein the chemotherapeutic agent is chosen from the group consisting of paclitaxel, adriamycin, cyclophosphamide, capecitabine, vinorelbine, carboplatin, epirubicin, docetaxel, methotrexate, 5-fluorouracil, anastrozole, letrozole, exemestane, or combinations thereof.

11. A method for decreasing the risk of endometrial hyperplasia or endometrial cancer associated with tamoxifen therapy, the method comprising administering a therapeutically effective amount of a composition chosen from the group consisting of soy germ, soy germ extract, isolated or synthetic daidzein, natural or synthetic equol, functionally equivalent derivatives or analogs of daidzein or equol, or combinations thereof to a patient undergoing a tamoxifen treatment regimen.

12. The method of claim 11 wherein the composition is chosen from the group consisting of isolated or synthetic daidzein, natural or synthetic equol, functionally equivalent derivatives or analogs of daidzein or equol, or combinations thereof.

13. The method of claim 11 wherein the composition comprises natural or synthetic equol.

14. The method of claim 11 further comprising administering a non-tamoxifen chemotherapeutic agent.

15. The method of claim 14 wherein the chemotherapeutic agent is chosen from the group consisting of paclitaxel, adriamycin, cyclophosphamide, capecitabine, vinorelbine, carboplatin, epirubicin, docetaxel, methotrexate, 5-fluorouracil, anastrozole, letrozole, exemestane, or combinations thereof.
